(19) United States
(12) Patent Application Publication
(10) Pub. No.: US 2018/0030372 A1
(43) Pub. Date: Feb. 1, 2018

(54) METHODS, DEVICES, AND SYSTEMS FOR EXTRACTION OF OILS FROM PLANT MATTER

(71) Applicants: Tapio Olavi Kristain MAKI, San Diego, CA (US); Jiri Eera lkari WICKSTROM, Helsinki (FI)

(72) Inventors: Tapio Olavi Kristain MAKI, San Diego, CA (US); Jiri Eera lkari WICKSTROM, Helsinki (FI)

(21) Appl. No.: 15/663,576

(22) Filed: Jul. 28, 2017

Related U.S. Application Data
(60) Provisional application No. 62/368,903, filed on Jul. 29, 2016.

(51) Int. Cl. C11B 1/10; C07C 53/02; C07C 31/08

(52) U.S. Cl. CPC C11B 1/10; C07C 31/08; C07C 53/02

(57) ABSTRACT

The present disclosure relates to methods, systems, and devices for efficiently extracting high quality oils and distillates, such as essential oils and hydrosols, from organic matter, such as plant matters. In some embodiments, organic matter comprises active chemical elements, which are used for therapeutic, medicinal, culinary, and industrial purposes.

### CROSS-REFERENCE

This application claims the benefit of U.S. Provisional Application No. 62/368,903 filed Jul. 29, 2016, which application is incorporated herein by reference.

### BACKGROUND

The present disclosure relates to methods, systems, and devices for efficiently extracting high quality oils and distillates, such as essential oils and hydrosols, from organic matter, such as plant matters. In some embodiments, the organic matter comprises active chemical elements, which are used for therapeutic, medicinal, industrial, and culinary purposes.

Although there are many current methods for extracting oils from organic matter, many of these methods are expensive, inefficient, and produce suboptimal products. Some such methods employ solvents such butane, hexane, and phenyl-phosphate that may leave unsafe residues which compromise the quality, effectiveness, and health benefits of the organic oil.

Although carbon dioxide (CO₂) extraction, which employs supercritical or subcritical CO₂ under high pressures and low temperatures, has been used to isolate organic oils and remove chlorophyll or other compounds, such methods may require expensive equipment, and significant fine tuning and adjustment to produce high quality oils.

Additionally, although solvents such as high grade grain alcohol, butanol, and olive oil have been used to extract organic oils, the use of such solvents in high volume production may be time-consuming and impractical.

As such, there is a current unmet need for methods, systems, and devices configured to efficiently extract high quality oils and distillates from an organic plant matter.

### SUMMARY

An aspect of the disclosure provides a device for producing an organic oil or a combination of organic oils from a plant matter comprising: a press which extracts the organic oil from the plant matter, wherein the press comprises: a compaction member; a compaction plate; a sieve; and an encapsulating member; a filter, wherein the filter is configured to filter the first solvent, the second solvent, or the organic oil; a distiller; and at least one of: an oil container, wherein the oil container retains a volume of the organic oil; or a liquid conveyor member, wherein the liquid conveyor member transfers a volume of the organic oil. In some embodiments, the device further comprises a cooling chamber which reduces a temperature of the plant matter, wherein the cooling chamber comprises: a refrigerator member; a conveyor member; and a first solvent dispersing member. In some embodiments, the cooling chamber further comprises cooling at least one of a first solvent dispersing member, a sealing door, a vacuum, and a regulated air intake.

In some embodiments, the refrigerator member is configured to maintain an air temperature inside the cooling chamber of about −125° C. to about 0° C. In some embodiments, the refrigerator member is configured to maintain an air temperature inside the cooling chamber of at least about −125° C. In some embodiments, the refrigerator member is configured to maintain an air temperature inside the cooling chamber of at most about 0° C. In some embodiments, the refrigerator member is configured to maintain an air temperature inside the cooling chamber of about 0° C. to about −10° C., about 0° C. to about −30° C., about 0° C. to about −40° C., about 0° C. to about −50° C., about 0° C. to about −50° C., about 0° C. to about −60° C., about 0° C. to about −70° C., about 0° C. to about −80° C., about 0° C. to about −90° C., about 0° C. to about −100° C., about 0° C. to about −125° C., about −10° C. to about −30° C., about −10° C. to about −40° C., about −10° C. to about −50° C., about −10° C. to about −50° C., about −10° C. to about −60° C., about −10° C. to about −70° C., about −10° C. to about −80° C., about −10° C. to about −90° C., about −10° C. to about −100° C., about −10° C. to about −125° C., about −30° C. to about −40° C., about −30° C. to about −50° C., about −30° C. to about −50° C., about −30° C. to about −60° C., about −30° C. to about −70° C., about −30° C. to about −80° C., about −30° C. to about −90° C., about −30° C. to about −100° C., about −30° C. to about −125° C., about −40° C. to about −50° C., about −40° C. to about −50° C., about −40° C. to about −60° C., about −40° C. to about −70° C., about −40° C. to about −80° C., about −40° C. to about −90° C., about −40° C. to about −100° C., about −40° C. to about −125° C., about −50° C. to about −50° C., about −50° C. to about −60° C., about −50° C. to about −70° C., about −50° C. to about −80° C., about −50° C. to about −90° C., about −50° C. to about −100° C., about −50° C. to about −125° C., about −50° C. to about −60° C., about −50° C. to about −70° C., about −50° C. to about −80° C., about −50° C. to about −90° C., about −50° C. to about −100° C., about −50° C. to about −125° C., about −60° C. to about −70° C., about −60° C. to about −80° C., about −60° C. to about −90° C., about −60° C. to about −100° C., about −60° C. to about −125° C., about −70° C. to about −80° C., about −70° C. to about −90° C., about −70° C. to about −100° C., about −70° C. to about −125° C., about −80° C. to about −90° C., about −80° C. to about −100° C., about −80° C. to about −125° C., about −90° C. to about −100° C., about −90° C. to about −125° C., or about −100° C. to about −125° C. In some embodiments, the refrigerator member is configured to maintain an air temperature inside the cooling chamber of about 0° C., about −10° C., about −30° C., about −40° C., about −50° C., about −50° C., about −60° C., about −70° C., about −80° C., about −90° C., about −100° C., or about −125° C.

In some embodiments, the conveyor member comprises a conveyor member, a slide, a roller, a crane, a forklift, a ramp, a vehicle, a lift, or any combination thereof. In some embodiments, the compaction plate comprises a plurality of protrusions. In some embodiments, the compaction plate and the second solvent dispersing member comprise a single component. In some embodiments, the compaction plate and the compaction member comprise a single component. In some embodiments, at least one of the first solvent dispersing member and the second solvent dispersing member comprises a nozzle, a dripper, a hose, a sprinkler, a faucet, a vent, or any combination thereof. In some embodiments, the sieve comprises a plurality of protrusions. In some embodiments, the encapsulating member is configured to temporarily surround the plant matter.

In some embodiments, the filter has a pore size of about 0.1 μm to about 10 μm. In some embodiments, the filter has a pore size of at least about 0.1 μm. In some embodiments, the filter has a pore size of at most about 10 μm. In some embodiments, the filter has a pore size of about 0.1 μm to about 0.2 μm, about 0.1 μm to about 0.5 μm, about 0.1 μm to about 1 μm, about 0.1 μm to about 2 μm, about 0.1 μm to about 3 μm, about 0.1 μm to about 4 μm, about 0.1 μm to about 5 μm, about 0.1 μm to about 6 μm, about 0.1 μm to about 7 μm, about 0.1 μm to about 8 μm, about 0.1 μm to about 10 μm, about 0.2 μm to about 0.5 μm, about 0.2 μm to about 1 μm, about 0.2 μm to about 2 μm, about 0.2 μm to about 3 μm, about 0.2 μm to about 4 μm, about 0.2 μm to about 5 μm, about 0.2 μm to about 6 μm, about 0.2 μm to about 7 μm, about 0.2 μm to about 8 μm, about 0.2 μm to about 10 μm, about 0.5 μm to about 1 μm, about 0.5 μm to about 2 μm, about 0.5 μm to about 3 μm, about 0.5 μm to about 4 μm, about 0.5 μm to about 5 μm, about 0.5 μm to about 6 μm, about 0.5 μm to about 7 μm, about 0.5 μm to about 8 μm, about 0.5 μm to about 10 μm, about 1 μm to about 2 μm, about 1 μm to about 3 μm, about 1 μm to about 4 μm, about 1 μm to about 5 μm, about 1 μm to about 6 μm, about 1 μm to about 7 μm, about 1 μm to about 8 μm, about 1 μm to about 10 μm, about 2 μm to about 3 μm, about 2 μm to about 4 μm, about 2 μm to about 5 μm, about 2 μm to about 6 μm, about 2 μm to about 7 μm, about 2 μm to about 8 μm, about 2 μm to about 10 μm, about 3 μm to about 4 μm, about 3 μm to about 5 μm, about 3 μm to about 6 μm, about 3 μm to about 7 μm, about 3 μm to about 8 μm, about 3 μm to about 10 μm, about 4 μm to about 5 μm, about 4 μm to about 6 μm, about 4 μm to about 7 μm, about 4 μm to about 8 μm, about 4 μm to about 10 μm, about 5 μm to about 6 μm, about 5 μm to about 7 μm, about 5 μm to about 8 μm, about 5 μm to about 10 μm, about 6 μm to about 7 μm, about 6 μm to about 8 μm, about 6 μm to about 10 μm, about 7 μm to about 8 μm, about 7 μm to about 10 μm, or about 8 μm to about 10 μm. In some embodiments, the filter has a pore size of about 0.1 μm, about 0.2 μm, about 0.5 μm, about 1 μm, about 2 μm, about 3 μm, about 4 μm, about 5 μm, about 6 μm, about 7 μm, about 8 μm, or about 10 μm.

In some embodiments, the device further comprises at least one of: a grinder, wherein the grinder is configured to reduce a size of the plant matter; and a vaporizing member, wherein the vaporizing member is configured to vaporize at least a portion of the organic oil. In some embodiments, the grinder comprises a blade, a grinding wheel, a saw, a disk, a wire cutter, or any combination thereof. In some embodiments, the device is configured to be mobile.

Another aspect provided herein is a method for extracting an organic oil from a plant matter comprising: cooling the plant matter; dispersing a first solvent onto the plant matter; and compressing the plant matter while dispersing a second solvent onto the plant matter.

In some embodiments, the plant matter is cooled to a temperature of about −125° C. to about 0° C. In some embodiments, the plant matter is cooled to a temperature of at least about −125° C. In some embodiments, the plant matter is cooled to a temperature of at most about 0° C. In some embodiments, the plant matter is cooled to a temperature of about 0° C. to about −10° C., about 0° C. to about −30° C., about 0° C. to about −40° C., about 0° C. to about −50° C., about 0° C. to about −50° C., about 0° C. to about −60° C., about 0° C. to about −70° C., about 0° C. to about −80° C., about 0° C. to about −90° C., about 0° C. to about −100° C., about 0° C. to about −125° C., about −10° C. to about −30° C., about −10° C. to about −40° C., about −10° C. to about −50° C., about −10° C. to about −50° C., about −10° C. to about −60° C., about −10° C. to about −70° C., about −10° C. to about −80° C., about −10° C. to about −90° C., about −10° C. to about −100° C., about −10° C. to about −125° C., about −30° C. to about −40° C., about −30° C. to about −50° C., about −30° C. to about −50° C., about −30° C. to about −60° C., about −30° C. to about −70° C., about −30° C. to about −80° C., about −30° C. to about −90° C., about −30° C. to about −100° C., about −30° C. to about −125° C., about −40° C. to about −50° C., about −40° C. to about −50° C., about −40° C. to about −60° C., about −40° C. to about −70° C., about −40° C. to about −80° C., about −40° C. to about −90° C., about −40° C. to about −100° C., about −40° C. to about −125° C., about −50° C. to about −50° C., about −50° C. to about −60° C., about −50° C. to about −70° C., about −50° C. to about −80° C., about −50° C. to about −90° C., about −50° C. to about −100° C., about −50° C. to about −125° C., about −50° C. to about −60° C., about −50° C. to about −70° C., about −50° C. to about −80° C., about −50° C. to about −90° C., about −50° C. to about −100° C., about −50° C. to about −125° C., about −60° C. to about −70° C., about −60° C. to about −80° C., about −60° C. to about −90° C., about −60° C. to about −100° C., about −60° C. to about −125° C., about −70° C. to about −80° C., about −70° C. to about −90° C., about −70° C. to about −100° C., about −70° C. to about −125° C., about −80° C. to about −90° C., about −80° C. to about −100° C., about −80° C. to about −125° C., about −90° C. to about −100° C., about −90° C. to about −125° C., or about −100° C. to about −125° C. In some embodiments, the plant matter is cooled to a temperature of about 0° C., about −10° C., about −30° C., about −40° C., about −50° C., about −50° C., about −60° C., about −70° C., about −80° C., about −90° C., about −100° C., or about −125° C.

In some embodiments, the mass of the plant matter is greater than the mass of the first solvent by a factor of about 1.01 to about 100. In some embodiments, the mass of the plant matter is greater than the mass of the first solvent by a factor of at least about 1.01. In some embodiments, the mass of the plant matter is greater than the mass of the first solvent by a factor of at most about 100. In some embodiments, the mass of the plant matter is greater than the mass of the first solvent by a factor of about 1.01 to about 1.5, about 1.01 to about 2, about 1.01 to about 5, about 1.01 to about 10, about 1.01 to about 20, about 1.01 to about 30, about 1.01 to about 40, about 1.01 to about 60, about 1.01 to about 80, about 1.01 to about 100, about 1.5 to about 2, about 1.5 to about 5, about 1.5 to about 10, about 1.5 to about 20, about 1.5 to about 30, about 1.5 to about 40, about 1.5 to about 60, about 1.5 to about 80, about 1.5 to about 100, about 2 to about 5, about 2 to about 10, about 2 to about 20, about 2 to about 30, about 2 to about 40, about 2 to about 60, about 2 to about 80, about 2 to about 100, about 5 to about 10, about 5 to about 20, about 5 to about 30, about 5 to about 40, about 5 to about 60, about 5 to about 80, about 5 to about 100, about 10 to about 20, about 10 to about 30, about 10 to about 40, about 10 to about 60, about 10 to about 80, about 10 to about 100, about 20 to about 30, about 20 to about 40, about 20 to about 60, about 20 to about 80, about 20 to about 100, about 30 to about 40, about 30 to about 60, about 30 to about 80, about 30 to about 100, about 40 to about 60, about 40 to about 80, about 40 to about 100, about 60 to about 80, about 60 to about 100, or about 80 to about 100. In some embodiments, the mass of the plant matter is greater than the mass of the first solvent by a factor of about 1.01, about 1.5, about 2, about 5, about 10, about 20, about 30, about 40, about 60, about 80, or about 100.

In some embodiments, the mass of the plant matter is greater than the mass of the second solvent by a factor of about 1.01 to about 100. In some embodiments, the mass of the plant matter is greater than the mass of the second solvent by a factor of at least about 1.01. In some embodiments, the mass of the plant matter is greater than the mass of the second solvent by a factor of at most about 100. In some embodiments, the mass of the plant matter is greater than the mass of the second solvent by a factor of about 1.01 to about 1.5, about 1.01 to about 2, about 1.01 to about 5, about 1.01 to about 10, about 1.01 to about 20, about 1.01 to about 30, about 1.01 to about 40, about 1.01 to about 60, about 1.01 to about 80, about 1.01 to about 100, about 1.5 to about 2, about 1.5 to about 5, about 1.5 to about 10, about 1.5 to about 20, about 1.5 to about 30, about 1.5 to about 40, about 1.5 to about 60, about 1.5 to about 80, about 1.5 to about 100, about 2 to about 5, about 2 to about 10, about 2 to about 20, about 2 to about 30, about 2 to about 40, about 2 to about 60, about 2 to about 80, about 2 to about 100, about 5 to about 10, about 5 to about 20, about 5 to about 30, about 5 to about 40, about 5 to about 60, about 5 to about 80, about 5 to about 100, about 10 to about 20, about 10 to about 30, about 10 to about 40, about 10 to about 60, about 10 to about 80, about 10 to about 100, about 20 to about 30, about 20 to about 40, about 20 to about 60, about 20 to about 80, about 20 to about 100, about 30 to about 40, about 30 to about 60, about 30 to about 80, about 30 to about 100, about 40 to about 60, about 40 to about 80, about 40 to about 100, about 60 to about 80, about 60 to about 100, or about 80 to about 100. In some embodiments, the mass of the plant matter is greater than the mass of the second solvent by a factor of about 1.01, about 1.5, about 2, about 5, about 10, about 20, about 30, about 40, about 60, about 80, or about 100.

In some embodiments, dispersing the first solvent onto the plant matter occurs at a pressure of about 1 bar to about 200 bar. In some embodiments, dispersing the first solvent onto the plant matter occurs at a pressure of at least about 1 bar. In some embodiments, dispersing the first solvent onto the plant matter occurs at a pressure of at most about 200 bar. In some embodiments, dispersing the first solvent onto the plant matter occurs at a pressure of about 1 bar to about 5 bar, about 1 bar to about 10 bar, about 1 bar to about 25 bar, about 1 bar to about 50 bar, about 1 bar to about 75 bar, about 1 bar to about 100 bar, about 1 bar to about 125 bar, about 1 bar to about 150 bar, about 1 bar to about 175 bar, about 1 bar to about 200 bar, about 5 bar to about 10 bar, about 5 bar to about 25 bar, about 5 bar to about 50 bar, about 5 bar to about 75 bar, about 5 bar to about 100 bar, about 5 bar to about 125 bar, about 5 bar to about 150 bar, about 5 bar to about 175 bar, about 5 bar to about 200 bar, about 10 bar to about 25 bar, about 10 bar to about 50 bar, about 10 bar to about 75 bar, about 10 bar to about 100 bar, about 10 bar to about 125 bar, about 10 bar to about 150 bar, about 10 bar to about 175 bar, about 10 bar to about 200 bar, about 25 bar to about 50 bar, about 25 bar to about 75 bar, about 25 bar to about 100 bar, about 25 bar to about 125 bar, about 25 bar to about 150 bar, about 25 bar to about 175 bar, about 25 bar to about 200 bar, about 50 bar to about 75 bar, about 50 bar to about 100 bar, about 50 bar to about 125 bar, about 50 bar to about 150 bar, about 50 bar to about 175 bar, about 50 bar to about 200 bar, about 75 bar to about 100 bar, about 75 bar to about 125 bar, about 75 bar to about 150 bar, about 75 bar to about 175 bar, about 75 bar to about 200 bar, about 100 bar to about 125 bar, about 100 bar to about 150 bar, about 100 bar to about 175 bar, about 100 bar to about 200 bar, about 125 bar to about 150 bar, about 125 bar to about 175 bar, about 125 bar to about 200 bar, about 150 bar to about 175 bar, about 150 bar to about 200 bar, or about 175 bar to about 200 bar. In some embodiments, dispersing the first solvent onto the plant matter occurs at a pressure of about 1 bar, about 5 bar, about 10 bar, about 25 bar, about 50 bar, about 75 bar, about 100 bar, about 125 bar, about 150 bar, about 175 bar, or about 200 bar.

In some embodiments, dispersing the second solvent onto the plant matter occurs at a pressure of about 1 bar to about 200 bar. In some embodiments, dispersing the second solvent onto the plant matter occurs at a pressure of at least about 1 bar. In some embodiments, dispersing the second solvent onto the plant matter occurs at a pressure of at most about 200 bar. In some embodiments, dispersing the second solvent onto the plant matter occurs at a pressure of about 1 bar to about 5 bar, about 1 bar to about 10 bar, about 1 bar to about 25 bar, about 1 bar to about 50 bar, about 1 bar to about 75 bar, about 1 bar to about 100 bar, about 1 bar to about 125 bar, about 1 bar to about 150 bar, about 1 bar to about 175 bar, about 1 bar to about 200 bar, about 5 bar to about 10 bar, about 5 bar to about 25 bar, about 5 bar to about 50 bar, about 5 bar to about 75 bar, about 5 bar to about 100 bar, about 5 bar to about 125 bar, about 5 bar to about 150 bar, about 5 bar to about 175 bar, about 5 bar to about 200 bar, about 10 bar to about 25 bar, about 10 bar to about 50 bar, about 10 bar to about 75 bar, about 10 bar to about 100 bar, about 10 bar to about 125 bar, about 10 bar to about 150 bar, about 10 bar to about 175 bar, about 10 bar to about 200 bar, about 25 bar to about 50 bar, about 25 bar to about 75 bar, about 25 bar to about 100 bar, about 25 bar to about 125 bar, about 25 bar to about 150 bar, about 25 bar to about 175 bar, about 25 bar to about 200 bar, about 50 bar to about 75 bar, about 50 bar to about 100 bar, about 50 bar to about 125 bar, about 50 bar to about 150 bar, about 50 bar to about 175 bar, about 50 bar to about 200 bar, about 75 bar to about 100 bar, about 75 bar to about 125 bar, about 75 bar to about 150 bar, about 75 bar to about 175 bar, about 75 bar to about 200 bar, about 100 bar to about 125 bar, about 100 bar to about 150 bar, about 100 bar to about 175 bar, about 100 bar to about 200 bar, about 125 bar to about 150 bar, about 125 bar to about 175 bar, about 125 bar to about 200 bar, about 150 bar to about 175 bar, about 150 bar to about 200 bar, or about 175 bar to about 200 bar. In some embodiments, dispersing the second solvent onto the plant matter occurs at a pressure of about 1 bar, about 5 bar, about 10 bar, about 25 bar, about 50 bar, about 75 bar, about 100 bar, about 125 bar, about 150 bar, about 175 bar, or about 200 bar.

In some embodiments, dispersing the first solvent onto the plant matter occurs over a period of time of about 2 seconds to about 500,000 seconds. In some embodiments, dispersing the first solvent onto the plant matter occurs over a period of time of at least about 2 seconds. In some embodiments, dispersing the first solvent onto the plant matter occurs over a period of time of at most about 500,000 seconds. In some embodiments, dispersing the first solvent onto the plant matter occurs over a period of time of about 2 seconds to about 5 seconds, about 2 seconds to about 10 seconds, about 2 seconds to about 50 seconds, about 2 seconds to about 100 seconds, about 2 seconds to about 500 seconds, about 2 seconds to about 1,000 seconds, about 2 seconds to about 5,000 seconds, about 2 seconds to about 10,000 seconds, about 2 seconds to about 50,000 seconds, about 2 seconds to about 100,000 seconds, about 2 seconds to about 500,000 seconds, about 5 seconds to about 10 seconds, about 5 seconds to about 50 seconds, about 5 seconds to about 100 seconds, about 5 seconds to about 500 seconds, about 5 seconds to about 1,000 seconds, about 5 seconds to about 5,000 seconds, about 5 seconds to about 10,000 seconds, about 5 seconds to about 50,000 seconds, about 5 seconds to about 100,000 seconds, about 5 seconds to about 500,000 seconds, about 10 seconds to about 50 seconds, about 10 seconds to about 100 seconds, about 10 seconds to about 500 seconds, about 10 seconds to about 1,000 seconds, about 10 seconds to about 5,000 seconds, about 10 seconds to about 10,000 seconds, about 10 seconds to about 50,000 seconds, about 10 seconds to about 100,000 seconds, about 10 seconds to about 500,000 seconds, about 50 seconds to about 100 seconds, about 50 seconds to about 500 seconds, about 50 seconds to about 1,000 seconds, about 50 seconds to about 5,000 seconds, about 50 seconds to about 10,000 seconds, about 50 seconds to about 50,000 seconds, about 50 seconds to about 100,000 seconds, about 50 seconds to about 500,000 seconds, about 100 seconds to about 500 seconds, about 100 seconds to about 1,000 seconds, about 100 seconds to about 5,000 seconds, about 100 seconds to about 10,000 seconds, about 100 seconds to about 50,000 seconds, about 100 seconds to about 100,000 seconds, about 100 seconds to about 500,000 seconds, about 500 seconds to about 1,000 seconds, about 500 seconds to about 5,000 seconds, about 500 seconds to about 10,000 seconds, about 500 seconds to about 50,000 seconds, about 500 seconds to about 100,000 seconds, about 500 seconds to about 500,000 seconds, about 1,000 seconds to about 5,000 seconds, about 1,000 seconds to about 10,000 seconds, about 1,000 seconds to about 50,000 seconds, about 1,000 seconds to about 100,000 seconds, about 1,000 seconds to about 500,000 seconds, about 5,000 seconds to about 10,000 seconds, about 5,000 seconds to about 50,000 seconds, about 5,000 seconds to about 100,000 seconds, about 5,000 seconds to about 500,000 seconds, about 10,000 seconds to about 50,000 seconds, about 10,000 seconds to about 100,000 seconds, about 10,000 seconds to about 500,000 seconds, about 50,000 seconds to about 100,000 seconds, about 50,000 seconds to about 500,000 seconds, or about 100,000 seconds to about 500,000 seconds. In some embodiments, dispersing the first solvent onto the plant matter occurs over a period of time of about 2 seconds, about 5 seconds, about 10 seconds, about 50 seconds, about 100 seconds, about 500 seconds, about 1,000 seconds, about 5,000 seconds, about 10,000 seconds, about 50,000 seconds, about 100,000 seconds, or about 500,000 seconds. In some embodiments, dispersing the first solvent onto the plant matter occurs over a period of time of more than 500,000 seconds.

In some embodiments, dispersing the second solvent onto the plant matter occurs over a period of time of about 2 seconds to about 500,000 seconds. In some embodiments, dispersing the second solvent onto the plant matter occurs over a period of time of at least about 2 seconds. In some embodiments, dispersing the second solvent onto the plant matter occurs over a period of time of at most about 500,000 seconds. In some embodiments, dispersing the second solvent onto the plant matter occurs over a period of time of about 2 seconds to about 5 seconds, about 2 seconds to about 10 seconds, about 2 seconds to about 50 seconds, about 2 seconds to about 100 seconds, about 2 seconds to about 500 seconds, about 2 seconds to about 1,000 seconds, about 2 seconds to about 5,000 seconds, about 2 seconds to about 10,000 seconds, about 2 seconds to about 50,000 seconds, about 2 seconds to about 100,000 seconds, about 2 seconds to about 500,000 seconds, about 5 seconds to about 10 seconds, about 5 seconds to about 50 seconds, about 5 seconds to about 100 seconds, about 5 seconds to about 500 seconds, about 5 seconds to about 1,000 seconds, about 5 seconds to about 5,000 seconds, about 5 seconds to about 10,000 seconds, about 5 seconds to about 50,000 seconds, about 5 seconds to about 100,000 seconds, about 5 seconds to about 500,000 seconds, about 10 seconds to about 50 seconds, about 10 seconds to about 100 seconds, about 10 seconds to about 500 seconds, about 10 seconds to about 1,000 seconds, about 10 seconds to about 5,000 seconds, about 10 seconds to about 10,000 seconds, about 10 seconds to about 50,000 seconds, about 10 seconds to about 100,000 seconds, about 10 seconds to about 500,000 seconds, about 50 seconds to about 100 seconds, about 50 seconds to about 500 seconds, about 50 seconds to about 1,000 seconds, about 50 seconds to about 5,000 seconds, about 50 seconds to about 10,000 seconds, about 50 seconds to about 50,000 seconds, about 50 seconds to about 100,000 seconds, about 50 seconds to about 500,000 seconds, about 100 seconds to about 500 seconds, about 100 seconds to about 1,000 seconds, about 100 seconds to about 5,000 seconds, about 100 seconds to about 10,000 seconds, about 100 seconds to about 50,000 seconds, about 100 seconds to about 100,000 seconds, about 100 seconds to about 500,000 seconds, about 500 seconds to about 1,000 seconds, about 500 seconds to about 5,000 seconds, about 500 seconds to about 10,000 seconds, about 500 seconds to about 50,000 seconds, about 500 seconds to about 100,000 seconds, about 500 seconds to about 500,000 seconds, about 1,000 seconds to about 5,000 seconds, about 1,000 seconds to about 10,000 seconds, about 1,000 seconds to about 50,000 seconds, about 1,000 seconds to about 100,000 seconds, about 1,000 seconds to about 500,000 seconds, about 5,000 seconds to about 10,000 seconds, about 5,000 seconds to about 50,000 seconds, about 5,000 seconds to about 100,000 seconds, about 5,000 seconds to about 500,000 seconds, about 10,000 seconds to about 50,000 seconds, about 10,000 seconds to about 100,000 seconds, about 10,000 seconds to about 500,000 seconds, about 50,000 seconds to about 100,000 seconds, about 50,000 seconds to about 500,000 seconds, or about 100,000 seconds to about 500,000 seconds. In some embodiments, dispersing the second solvent onto the plant matter occurs over a period of time of about 2 seconds, about 5 seconds, about 10 seconds, about 50 seconds, about 100 seconds, about 500 seconds, about 1,000 seconds, about 5,000 seconds, about 10,000 seconds, about 50,000 seconds, about 100,000 seconds, or about 500,000 seconds. In some embodiments, dispersing the second solvent onto the plant matter occurs over a period of time of more than 500,000 seconds.

In some embodiments, compressing the plant matter occurs over a period of time of about 1 minute to about 60 minutes

In some embodiments, compressing the plant matter occurs over a period of time of about 2 seconds to about 500,000 seconds. In some embodiments, compressing the plant matter occurs over a period of time of at least about 2 seconds. In some embodiments, compressing the plant matter occurs over a period of time of at most about 500,000 seconds. In some embodiments, compressing the plant matter occurs over a period of time of about 2 seconds to about 5 seconds, about 2 seconds to about 10 seconds, about 2 seconds to about 50 seconds, about 2 seconds to about 100 seconds, about 2 seconds to about 500 seconds, about 2 seconds to about 1,000 seconds, about 2 seconds to about 5,000 seconds, about 2 seconds to about 10,000 seconds, about 2 seconds to about 50,000 seconds, about 2 seconds to about 100,000 seconds, about 2 seconds to about 500,000 seconds, about 5 seconds to about 10 seconds, about 5 seconds to about 50 seconds, about 5 seconds to about 100 seconds, about 5 seconds to about 500 seconds, about 5 seconds to about 1,000 seconds, about 5 seconds to about 5,000 seconds, about 5 seconds to about 10,000 seconds, about 5 seconds to about 50,000 seconds, about 5 seconds to about 100,000 seconds, about 5 seconds to about 500,000 seconds, about 10 seconds to about 50 seconds, about 10 seconds to about 100 seconds, about 10 seconds to about 500 seconds, about 10 seconds to about 1,000 seconds, about 10 seconds to about 5,000 seconds, about 10 seconds to about 10,000 seconds, about 10 seconds to about 50,000 seconds, about 10 seconds to about 100,000 seconds, about 10 seconds to about 500,000 seconds, about 50 seconds to about 100 seconds, about 50 seconds to about 500 seconds, about 50 seconds to about 1,000 seconds, about 50 seconds to about 5,000 seconds, about 50 seconds to about 10,000 seconds, about 50 seconds to about 50,000 seconds, about 50 seconds to about 100,000 seconds, about 50 seconds to about 500,000 seconds, about 100 seconds to about 500 seconds, about 100 seconds to about 1,000 seconds, about 100 seconds to about 5,000 seconds, about 100 seconds to about 10,000 seconds, about 100 seconds to about 50,000 seconds, about 100 seconds to about 100,000 seconds, about 100 seconds to about 500,000 seconds, about 500 seconds to about 1,000 seconds, about 500 seconds to about 5,000 seconds, about 500 seconds to about 10,000 seconds, about 500 seconds to about 50,000 seconds, about 500 seconds to about 100,000 seconds, about 500 seconds to about 500,000 seconds, about 1,000 seconds to about 5,000 seconds, about 1,000 seconds to about 10,000 seconds, about 1,000 seconds to about 50,000 seconds, about 1,000 seconds to about 100,000 seconds, about 1,000 seconds to about 500,000 seconds, about 5,000 seconds to about 10,000 seconds, about 5,000 seconds to about 50,000 seconds, about 5,000 seconds to about 100,000 seconds, about 5,000 seconds to about 500,000 seconds, about 10,000 seconds to about 50,000 seconds, about 10,000 seconds to about 100,000 seconds, about 10,000 seconds to about 500,000 seconds, about 50,000 seconds to about 100,000 seconds, about 50,000 seconds to about 500,000 seconds, or about 100,000 seconds to about 500,000 seconds. In some embodiments, compressing the plant matter occurs over a period of time of about 2 seconds, about 5 seconds, about 10 seconds, about 50 seconds, about 100 seconds, about 500 seconds, about 1,000 seconds, about 5,000 seconds, about 10,000 seconds, about 50,000 seconds, about 100,000 seconds, or about 500,000 seconds.

In some embodiments, compressing the plant matter reduces the volume of the plant matter by about 1% to about 95%. In some embodiments, compressing the plant matter reduces the volume of the plant matter by at least about 1%. In some embodiments, compressing the plant matter reduces the volume of the plant matter by at most about 95%. In some embodiments, compressing the plant matter reduces the volume of the plant matter by about 1% to about 2%, about 1% to about 5%, about 1% to about 10%, about 1% to about 20%, about 1% to about 30%, about 1% to about 40%, about 1% to about 50%, about 1% to about 60%, about 1% to about 70%, about 1% to about 80%, about 1% to about 95%, about 2% to about 5%, about 2% to about 10%, about 2% to about 20%, about 2% to about 30%, about 2% to about 40%, about 2% to about 50%, about 2% to about 60%, about 2% to about 70%, about 2% to about 80%, about 2% to about 95%, about 5% to about 10%, about 5% to about 20%, about 5% to about 30%, about 5% to about 40%, about 5% to about 50%, about 5% to about 60%, about 5% to about 70%, about 5% to about 80%, about 5% to about 95%, about 10% to about 20%, about 10% to about 30%, about 10% to about 40%, about 10% to about 50%, about 10% to about 60%, about 10% to about 70%, about 10% to about 80%, about 10% to about 95%, about 20% to about 30%, about 20% to about 40%, about 20% to about 50%, about 20% to about 60%, about 20% to about 70%, about 20% to about 80%, about 20% to about 95%, about 30% to about 40%, about 30% to about 50%, about 30% to about 60%, about 30% to about 70%, about 30% to about 80%, about 30% to about 95%, about 40% to about 50%, about 40% to about 60%, about 40% to about 70%, about 40% to about 80%, about 40% to about 95%, about 50% to about 60%, about 50% to about 70%, about 50% to about 80%, about 50% to about 95%, about 60% to about 70%, about 60% to about 80%, about 60% to about 95%, about 70% to about 80%, about 70% to about 95%, or about 80% to about 95%. In some embodiments, compressing the plant matter reduces the volume of the plant matter by about 1%, about 2%, about 5%, about 10%, about 20%, about 30%, about 40%, about 50%, about 60%, about 70%, about 80%, or about 95%.

Some embodiments further comprise heating the plant matter before cooling the plant matter.

In some embodiments, the plant matter is heated to a temperature of about 50° C. to about 200° C. In some embodiments, the plant matter is heated to a temperature of at least about 50° C. In some embodiments, the plant matter is heated to a temperature of at most about 200° C. In some embodiments, the plant matter is heated to a temperature of about 50° C. to about 60° C., about 50° C. to about 70° C., about 50° C. to about 80° C., about 50° C. to about 90° C., about 50° C. to about 100° C., about 50° C. to about 125° C., about 50° C. to about 150° C., about 50° C. to about 175° C., about 50° C. to about 200° C., about 60° C. to about 70° C., about 60° C. to about 80° C., about 60° C. to about 90° C., about 60° C. to about 100° C., about 60° C. to about 125° C., about 60° C. to about 150° C., about 60° C. to about 175° C., about 60° C. to about 200° C., about 70° C. to about 80° C., about 70° C. to about 90° C., about 70° C. to about 100° C., about 70° C. to about 125° C., about 70° C. to about 150° C., about 70° C. to about 175° C., about 70° C. to about 200° C., about 80° C. to about 90° C., about 80° C. to about 100° C., about 80° C. to about 125° C., about 80° C. to about 150° C., about 80° C. to about 175° C., about 80° C. to about 200° C., about 90° C. to about 100° C., about 90° C. to about 125° C., about 90° C. to about 150° C., about 90° C. to about 175° C., about 90° C. to about 200° C., about 100° C. to about 125° C., about 100° C. to about 150° C., about 100° C. to about 175° C., about 100° C. to about 200° C., about 125° C. to about 150° C., about 125° C. to about 175° C., about 125° C. to about 200° C., about 150° C. to about 175° C., about 150° C. to about 200° C., or about 175° C. to about 200° C. In some embodiments, the plant matter is heated to a temperature of about 50° C., about 60° C., about 70° C., about 80° C., about 90° C., about 100° C., about 125° C., about 150° C., about 175° C., or about 200° C.

In some embodiments, the plant matter is heated for a period of time of about 1 hour to about 24 hours. In some embodiments, the plant matter is heated for a period of time of at least about 1 hour. In some embodiments, the plant matter is heated for a period of time of at most about 24 hours. In some embodiments, the plant matter is heated for a period of time of about 1 hour to about 2 hours, about 1 hour to about 4 hours, about 1 hour to about 6 hours, about 1 hour to about 8 hours, about 1 hour to about 10 hours, about 1 hour to about 14 hours, about 1 hour to about 18 hours, about 1 hour to about 24 hours, about 2 hours to about 4 hours, about 2 hours to about 6 hours, about 2 hours to about 8 hours, about 2 hours to about 10 hours, about 2 hours to about 14 hours, about 2 hours to about 18 hours, about 2 hours to about 24 hours, about 4 hours to about 6 hours, about 4 hours to about 8 hours, about 4 hours to about 10 hours, about 4 hours to about 14 hours, about 4 hours to about 18 hours, about 4 hours to about 24 hours, about 6 hours to about 8 hours, about 6 hours to about 10 hours, about 6 hours to about 14 hours, about 6 hours to about 18 hours, about 6 hours to about 24 hours, about 8 hours to about 10 hours, about 8 hours to about 14 hours, about 8 hours to about 18 hours, about 8 hours to about 24 hours, about 10 hours to about 14 hours, about 10 hours to about 18 hours, about 10 hours to about 24 hours, about 14 hours to about 18 hours, about 14 hours to about 24 hours, or about 18 hours to about 24 hours. In some embodiments, the plant matter is heated for a period of time of about 1 hour, about 2 hours, about 4 hours, about 6 hours, about 8 hours, about 10 hours, about 14 hours, about 18 hours, or about 24 hours.

Some embodiments further comprise grinding the plant matter before compressing the plant matter, wherein grinding the plant matter reduces a size of an aggregate size of the plant matter. Some embodiments further comprise filtering the organic oil.

In some embodiments, filtering the organic oil comprises filtering the organic oil through a filter with a pore size of about 0.1 μm to about 10 μm. In some embodiments, filtering the organic oil comprises filtering the organic oil through a filter with a pore size of at least about 0.1 μm. In some embodiments, filtering the organic oil comprises filtering the organic oil through a filter with a pore size of at most about 10 μm. In some embodiments, filtering the organic oil comprises filtering the organic oil through a filter with a pore size of about 0.1 μm to about 0.2 μm, about 0.1 μm to about 0.5 μm, about 0.1 μm to about 1 μm, about 0.1 μm to about 2 μm, about 0.1 μm to about 3 μm, about 0.1 μm to about 4 μm, about 0.1 μm to about 5 μm, about 0.1 μm to about 6 μm, about 0.1 μm to about 7 μm, about 0.1 μm to about 8 μm, about 0.1 μm to about 10 μm, about 0.2 μm to about 0.5 μm, about 0.2 μm to about 1 μm, about 0.2 μm to about 2 μm, about 0.2 μm to about 3 μm, about 0.2 μm to about 4 μm, about 0.2 μm to about 5 μm, about 0.2 μm to about 6 μm, about 0.2 μm to about 7 μm, about 0.2 μm to about 8 μm, about 0.2 μm to about 10 μm, about 0.5 μm to about 1 μm, about 0.5 μm to about 2 μm, about 0.5 μm to about 3 μm, about 0.5 μm to about 4 μm, about 0.5 μm to about 5 μm, about 0.5 μm to about 6 μm, about 0.5 μm to about 7 μm, about 0.5 μm to about 8 μm, about 0.5 μm to about 10 μm, about 1 μm to about 2 μm, about 1 μm to about 3 μm, about 1 μm to about 4 μm, about 1 μm to about 5 μm, about 1 μm to about 6 μm, about 1 μm to about 7 μm, about 1 μm to about 8 μm, about 1 μm to about 10 μm, about 2 μm to about 3 μm, about 2 μm to about 4 μm, about 2 μm to about 5 μm, about 2 μm to about 6 μm, about 2 μm to about 7 μm, about 2 μm to about 8 μm, about 2 μm to about 10 μm, about 3 μm to about 4 μm, about 3 μm to about 5 μm, about 3 μm to about 6 μm, about 3 μm to about 7 μm, about 3 μm to about 8 μm, about 3 μm to about 10 μm, about 4 μm to about 5 μm, about 4 μm to about 6 μm, about 4 μm to about 7 μm, about 4 μm to about 8 μm, about 4 μm to about 10 μm, about 5 μm to about 6 μm, about 5 μm to about 7 μm, about 5 μm to about 8 μm, about 5 μm to about 10 μm, about 6 μm to about 7 μm, about 6 μm to about 8 μm, about 6 μm to about 10 μm, about 7 μm to about 8 μm, about 7 μm to about 10 μm, or about 8 μm to about 10 μm. In some embodiments, filtering the organic oil comprises filtering the organic oil through a filter with a pore size of about 0.1 μm, about 0.2 μm, about 0.5 μm, about 1 μm, about 2 μm, about 3 μm, about 4 μm, about 5 μm, about 6 μm, about 7 μm, about 8 μm, or about 10 μm.

Some embodiments further comprise adding water to the organic oil.

In some embodiments, the volume of the organic oil is greater than the volume of the water by a factor of about 1.1 to about 10.

In some embodiments, the volume of the organic oil is greater than the volume of the water by a factor of about 1.01 to about 100. In some embodiments, the volume of the organic oil is greater than the volume of the water by a factor of at least about 1.01. In some embodiments, the volume of the organic oil is greater than the volume of the water by a factor of at most about 100. In some embodiments, the volume of the organic oil is greater than the volume of the water by a factor of about 1.01 to about 1.5, about 1.01 to about 2, about 1.01 to about 5, about 1.01 to about 10, about 1.01 to about 20, about 1.01 to about 30, about 1.01 to about 40, about 1.01 to about 60, about 1.01 to about 80, about 1.01 to about 100, about 1.5 to about 2, about 1.5 to about 5, about 1.5 to about 10, about 1.5 to about 20, about 1.5 to about 30, about 1.5 to about 40, about 1.5 to about 60, about 1.5 to about 80, about 1.5 to about 100, about 2 to about 5, about 2 to about 10, about 2 to about 20, about 2 to about 30, about 2 to about 40, about 2 to about 60, about 2 to about 80, about 2 to about 100, about 5 to about 10, about 5 to about 20, about 5 to about 30, about 5 to about 40, about 5 to about 60, about 5 to about 80, about 5 to about 100, about 10 to about 20, about 10 to about 30, about 10 to about 40, about 10 to about 60, about 10 to about 80, about 10 to about 100, about 20 to about 30, about 20 to about 40, about 20 to about 60, about 20 to about 80, about 20 to about 100, about 30 to about 40, about 30 to about 60, about 30 to about 80, about 30 to about 100, about 40 to about 60, about 40 to about 80, about 40 to about 100, about 60 to about 80, about 60 to about 100, or about 80 to about 100. In some embodiments, the volume of the organic oil is greater than the volume of the water by a factor of about 1.01, about 1.5, about 2, about 5, about 10, about 20, about 30, about 40, about 60, about 80, or about 100.

Some embodiments further comprise distilling the organic oil. In some embodiments, distilling the organic oil comprises vacuum distillation, molecular distillation, steam distillation, evaporation, or any combination thereof.

In some embodiments, distilling the organic oil comprises evaporation, and wherein the evaporation comprises heating the organic oil at a temperature of about 50° C. to about 100° C. In some embodiments, distilling the organic oil comprises evaporation, and wherein the evaporation comprises heating the organic oil at a temperature of at least about 50° C. In some embodiments, distilling the organic oil comprises evaporation, and wherein the evaporation comprises heating the organic oil at a temperature of at most about 100° C. In some embodiments, distilling the organic oil comprises evaporation, and wherein the evaporation comprises heating the organic oil at a temperature of about 50° C. to about 55° C., about 50° C. to about 60° C., about 50° C. to about 65° C., about 50° C. to about 70° C., about 50° C. to about 75° C., about 50° C. to about 80° C., about 50° C. to about 85° C., about 50° C. to about 90° C., about 50° C. to about 95° C., about 50° C. to about 100° C., about 55° C. to about 60° C., about 55° C. to about 65° C., about 55° C. to about 70° C., about 55° C. to about 75° C., about 55° C. to about 80° C., about 55° C. to about 85° C., about 55° C. to about 90° C., about 55° C. to about 95° C., about 55° C. to about 100° C., about 60° C. to about 65° C., about 60° C. to about 70° C., about 60° C. to about 75° C., about 60° C. to about 80° C., about 60° C. to about 85° C., about 60° C. to about 90° C., about 60° C. to about 95° C., about 60° C. to about 100° C., about 65° C. to about 70° C., about 65° C. to about 75° C., about 65° C. to about 80° C., about 65° C. to about 85° C., about 65° C. to about 90° C., about 65° C. to about 95° C., about 65° C. to about 100° C., about 70° C. to about 75° C., about 70° C. to about 80° C., about 70° C. to about 85° C., about 70° C. to about 90° C., about 70° C. to about 95° C., about 70° C. to about 100° C., about 75° C. to about 80° C., about 75° C. to about 85° C., about 75° C. to about 90° C., about 75° C. to about 95° C., about 75° C. to about 100° C., about 80° C. to about 85° C., about 80° C. to about 90° C., about 80° C. to about 95° C., about 80° C. to about 100° C., about 85° C. to about 90° C., about 85° C. to about 95° C., about 85° C. to about 100° C., about 90° C. to about 95° C., about 90° C. to about 100° C., or about 95° C. to about 100° C. In some embodiments, distilling the organic oil comprises evaporation, and wherein the evaporation comprises heating the organic oil at a temperature of about 50° C., about 55° C., about 60° C., about 65° C., about 70° C., about 75° C., about 80° C., about 85° C., about 90° C., about 95° C., or about 100° C.

In some embodiments, distilling the organic oil comprises steam distillation, and wherein the steam distillation comprises heating the organic oil at a temperature of about 50° C. to about 100° C. In some embodiments, distilling the organic oil comprises steam distillation, and wherein the steam distillation comprises heating the organic oil at a temperature of at least about 50° C. In some embodiments, distilling the organic oil comprises steam distillation, and wherein the steam distillation comprises heating the organic oil at a temperature of at most about 100° C. In some embodiments, distilling the organic oil comprises steam distillation, and wherein the steam distillation comprises heating the organic oil at a temperature of about 50° C. to about 55° C., about 50° C. to about 60° C., about 50° C. to about 65° C., about 50° C. to about 70° C., about 50° C. to about 75° C., about 50° C. to about 80° C., about 50° C. to about 85° C., about 50° C. to about 90° C., about 50° C. to about 95° C., about 50° C. to about 100° C., about 55° C. to about 60° C., about 55° C. to about 65° C., about 55° C. to about 70° C., about 55° C. to about 75° C., about 55° C. to about 80° C., about 55° C. to about 85° C., about 55° C. to about 90° C., about 55° C. to about 95° C., about 55° C. to about 100° C., about 60° C. to about 65° C., about 60° C. to about 70° C., about 60° C. to about 75° C., about 60° C. to about 80° C., about 60° C. to about 85° C., about 60° C. to about 90° C., about 60° C. to about 95° C., about 60° C. to about 100° C., about 65° C. to about 70° C., about 65° C. to about 75° C., about 65° C. to about 80° C., about 65° C. to about 85° C., about 65° C. to about 90° C., about 65° C. to about 95° C., about 65° C. to about 100° C., about 70° C. to about 75° C., about 70° C. to about 80° C., about 70° C. to about 85° C., about 70° C. to about 90° C., about 70° C. to about 95° C., about 70° C. to about 100° C., about 75° C. to about 80° C., about 75° C. to about 85° C., about 75° C. to about 90° C., about 75° C. to about 95° C., about 75° C. to about 100° C., about 80° C. to about 85° C., about 80° C. to about 90° C., about 80° C. to about 95° C., about 80° C. to about 100° C., about 85° C. to about 90° C., about 85° C. to about 95° C., about 85° C. to about 100° C., about 90° C. to about 95° C., about 90° C. to about 100° C., or about 95° C. to about 100° C. In some embodiments, distilling the organic oil comprises steam distillation, and wherein the steam distillation comprises heating the organic oil at a temperature of about 50° C., about 55° C., about 60° C., about 65° C., about 70° C., about 75° C., about 80° C., about 85° C., about 90° C., about 95° C., or about 100° C.

In some embodiments, distilling the organic oil comprises evaporation, wherein the evaporation comprises heating the organic oil for a period of time of about 0.5 hours to about 6 hours. In some embodiments, distilling the organic oil comprises evaporation, wherein the evaporation comprises heating the organic oil for a period of time of at least about 0.5 hours. In some embodiments, distilling the organic oil comprises evaporation, wherein the evaporation comprises heating the organic oil for a period of time of at most about 6 hours. In some embodiments, distilling the organic oil comprises evaporation, wherein the evaporation comprises heating the organic oil for a period of time of about 0.5 hours to about 1 hour, about 0.5 hours to about 1.5 hours, about 0.5 hours to about 2 hours, about 0.5 hours to about 2.5 hours, about 0.5 hours to about 3 hours, about 0.5 hours to about 3.5 hours, about 0.5 hours to about 4 hours, about 0.5 hours to about 4.5 hours, about 0.5 hours to about 5 hours, about 0.5 hours to about 5.5 hours, about 0.5 hours to about 6 hours, about 1 hour to about 1.5 hours, about 1 hour to about 2 hours, about 1 hour to about 2.5 hours, about 1 hour to about 3 hours, about 1 hour to about 3.5 hours, about 1 hour to about 4 hours, about 1 hour to about 4.5 hours, about 1 hour to about 5 hours, about 1 hour to about 5.5 hours, about 1 hour to about 6 hours, about 1.5 hours to about 2 hours, about 1.5 hours to about 2.5 hours, about 1.5 hours to about 3 hours, about 1.5 hours to about 3.5 hours, about 1.5 hours to about 4 hours, about 1.5 hours to about 4.5 hours, about 1.5 hours to about 5 hours, about 1.5 hours to about 5.5 hours, about 1.5 hours to about 6 hours, about 2 hours to about 2.5 hours, about 2 hours to about 3 hours, about 2 hours to about 3.5 hours, about 2 hours to about 4 hours, about 2 hours to about 4.5 hours, about 2 hours to about 5 hours, about 2 hours to about 5.5 hours, about 2 hours to about 6 hours, about 2.5 hours to about 3 hours, about 2.5 hours to about 3.5 hours, about 2.5 hours to about 4 hours, about 2.5 hours to about 4.5 hours, about 2.5 hours to about 5 hours, about 2.5 hours to about 5.5 hours, about 2.5 hours to about 6 hours, about 3 hours to about 3.5 hours, about 3 hours to about 4 hours, about 3 hours to about 4.5 hours, about 3 hours to about 5 hours, about 3 hours to about 5.5 hours, about 3 hours to about 6 hours, about 3.5 hours to about 4 hours, about 3.5 hours to about 4.5 hours, about 3.5 hours to about 5 hours, about 3.5 hours to about 5.5 hours, about 3.5 hours to about 6 hours, about 4 hours to about 4.5 hours, about 4 hours to about 5 hours, about 4 hours to about 5.5 hours, about 4 hours to about 6 hours, about 4.5 hours to about 5 hours, about 4.5 hours to about 5.5 hours, about 4.5 hours to about 6 hours, about 5 hours to about 5.5 hours, about 5 hours to about 6 hours, or about 5.5 hours to about 6 hours. In some embodiments, distilling the organic oil comprises evaporation, wherein the evaporation comprises heating the organic oil for a period of time of about 0.5 hours, about 1 hour, about 1.5 hours, about 2 hours, about 2.5 hours, about 3 hours, about 3.5 hours, about 4 hours, about 4.5 hours, about 5 hours, about 5.5 hours, or about 6 hours.

In some embodiments, distilling the organic oil occurs over a period of time of about 1 hour to about 1 hour to about 1,400 hours. In some embodiments, distilling the organic oil occurs over a period of time of about 1 hour to at least about 1 hour. In some embodiments, distilling the organic oil occurs over a period of time of about 1 hour to at most about 1,400 hours. In some embodiments, distilling the organic oil occurs over a period of time of about 1 hour to about 1 hour to about 2 hours, about 1 hour to about 5 hours, about 1 hour to about 10 hours, about 1 hour to about 20 hours, about 1 hour to about 50 hours, about 1 hour to about 100 hours, about 1 hour to about 200 hours, about 1 hour to about 500 hours, about 1 hour to about 1,000 hours, about 1 hour to about 1,400 hours, about 2 hours to about 5 hours, about 2 hours to about 10 hours, about 2 hours to about 20 hours, about 2 hours to about 50 hours, about 2 hours to about 100 hours, about 2 hours to about 200 hours, about 2 hours to about 500 hours, about 2 hours to about 1,000 hours, about 2 hours to about 1,400 hours, about 5 hours to about 10 hours, about 5 hours to about 20 hours, about 5 hours to about 50 hours, about 5 hours to about 100 hours, about 5 hours to about 200 hours, about 5 hours to about 500 hours, about 5 hours to about 1,000 hours, about 5 hours to about 1,400 hours, about 10 hours to about 20 hours, about 10 hours to about 50 hours, about 10 hours to about 100 hours, about 10 hours to about 200 hours, about 10 hours to about 500 hours, about 10 hours to about 1,000 hours, about 10 hours to about 1,400 hours, about 20 hours to about 50 hours, about 20 hours to about 100 hours, about 20 hours to about 200 hours, about 20 hours to about 500 hours, about 20 hours to about 1,000 hours, about 20 hours to about 1,400 hours, about 50 hours to about 100 hours, about 50 hours to about 200 hours, about 50 hours to about 500 hours, about 50 hours to about 1,000 hours, about 50 hours to about 1,400 hours, about 100 hours to about 200 hours, about 100 hours to about 500 hours, about 100 hours to about 1,000 hours, about 100 hours to about 1,400 hours, about 200 hours to about 500 hours, about 200 hours to about 1,000 hours, about 200 hours to about 1,400 hours, about 500 hours to about 1,000 hours, about 500 hours to about 1,400 hours, or about 1,000 hours to about 1,400 hours. In some embodiments, distilling the organic oil occurs over a period of time of about 1 hour to about 1 hour, about 2 hours, about 5 hours, about 10 hours, about 20 hours, about 50 hours, about 100 hours, about 200 hours, about 500 hours, about 1,000 hours, or about 1,400 hours.

Some embodiments further comprise compacting the plant matter before dispersing the second solvent, wherein compacting the plant matter reduces a volume of the plant matter. Some embodiments further comprise recycling at least one of the first solvent and the second solvent from a first batch to a subsequent batch. Some embodiments further comprise vaporizing the organic oil. In some embodiments, vaporizing comprises ice drying, air pressure drying, convection drying, conduction drying, or any combination thereof.

In some embodiments, the form of the plant matter comprises a bale, wherein each bale has a weight of about 100 kg to about 400 kg. In some embodiments, the form of the plant matter comprises a bale, wherein each bale has a weight of at least about 100 kg. In some embodiments, the form of the plant matter comprises a bale, wherein each bale has a weight of at most about 400 kg. In some embodiments, the form of the plant matter comprises a bale, wherein each bale has a weight of about 100 kg to about 150 kg, about 100 kg to about 200 kg, about 100 kg to about 250 kg, about 100 kg to about 300 kg, about 100 kg to about 350 kg, about 100 kg to about 400 kg, about 150 kg to about 200 kg, about 150 kg to about 250 kg, about 150 kg to about 300 kg, about 150 kg to about 350 kg, about 150 kg to about 400 kg, about 200 kg to about 250 kg, about 200 kg to about 300 kg, about 200 kg to about 350 kg, about 200 kg to about 400 kg, about 250 kg to about 300 kg, about 250 kg to about 350 kg, about 250 kg to about 400 kg, about 300 kg to about 350 kg, about 300 kg to about 400 kg, or about 350 kg to about 400 kg. In some embodiments, the form of the plant matter comprises a bale, wherein each bale has a weight of about 100 kg, about 150 kg, about 200 kg, about 250 kg, about 300 kg, about 350 kg, or about 400 kg.

In some embodiments, the form of the plant matter comprises a bale, wherein each bale has a diameter of about 0.5 m to about 4 m. In some embodiments, the form of the plant matter comprises a bale, wherein each bale has a diameter of at least about 0.5 m. In some embodiments, the form of the plant matter comprises a bale, wherein each bale has a diameter of at most about 4 m. In some embodiments, the form of the plant matter comprises a bale, wherein each bale has a diameter of about 0.5 m to about 1 m, about 0.5 m to about 1.5 m, about 0.5 m to about 2 m, about 0.5 m to about 2.5 m, about 0.5 m to about 3 m, about 0.5 m to about 3.5 m, about 0.5 m to about 4 m, about 1 m to about 1.5 m, about 1 m to about 2 m, about 1 m to about 2.5 m, about 1 m to about 3 m, about 1 m to about 3.5 m, about 1 m to about 4 m, about 1.5 m to about 2 m, about 1.5 m to about 2.5 m, about 1.5 m to about 3 m, about 1.5 m to about 3.5 m, about 1.5 m to about 4 m, about 2 m to about 2.5 m, about 2 m to about 3 m, about 2 m to about 3.5 m, about 2 m to about 4 m, about 2.5 m to about 3 m, about 2.5 m to about 3.5 m, about 2.5 m to about 4 m, about 3 m to about 3.5 m, about 3 m to about 4 m, or about 3.5 m to about 4 m. In some embodiments, the form of the plant matter comprises a bale, wherein each bale has a diameter of about 0.5 m, about 1 m, about 1.5 m, about 2 m, about 2.5 m, about 3 m, about 3.5 m, or about 4 m.

In some embodiments, the form of the plant matter comprises a bale, wherein each bale has a height of about 0.5 m to about 4 m. In some embodiments, the form of the plant matter comprises a bale, wherein each bale has a height of at least about 0.5 m. In some embodiments, the form of the plant matter comprises a bale, wherein each bale has a height of at most about 4 m. In some embodiments, the form of the plant matter comprises a bale, wherein each bale has a height of about 0.5 m to about 1 m, about 0.5 m to about 1.5 m, about 0.5 m to about 2 m, about 0.5 m to about 2.5 m, about 0.5 m to about 3 m, about 0.5 m to about 3.5 m, about 0.5 m to about 4 m, about 1 m to about 1.5 m, about 1 m to about 2 m, about 1 m to about 2.5 m, about 1 m to about 3 m, about 1 m to about 3.5 m, about 1 m to about 4 m, about 1.5 m to about 2 m, about 1.5 m to about 2.5 m, about 1.5 m to about 3 m, about 1.5 m to about 3.5 m, about 1.5 m to about 4 m, about 2 m to about 2.5 m, about 2 m to about 3 m, about 2 m to about 3.5 m, about 2 m to about 4 m, about 2.5 m to about 3 m, about 2.5 m to about 3.5 m, about 2.5 m to about 4 m, about 3 m to about 3.5 m, about 3 m to about 4 m, or about 3.5 m to about 4 m. In some embodiments, the form of the plant matter comprises a bale, wherein each bale has a height of about 0.5 m, about 1 m, about 1.5 m, about 2 m, about 2.5 m, about 3 m, about 3.5 m, or about 4 m. In some embodiments the bale comprises plant matter that is packed closely or loosely together.

Other goals and advantages of the disclosure will be further appreciated and understood when considered in conjunction with the following description and accompanying drawings. While the following description may contain specific details describing particular embodiments of the disclosure, this should not be construed as limitations to the scope of the disclosure but rather as an exemplification of preferable embodiments. For each aspect of the disclosure, many variations are possible as suggested herein that are known to those of ordinary skill in the art. A variety of changes and modifications can be made within the scope of the disclosure without departing from the spirit thereof.

### BRIEF DESCRIPTION OF THE DRAWINGS

The features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings or figures (also “FIG.” and “FIGS.” herein), of which:

FIG. 1A shows a cross-sectioned side view of an exemplary device for extracting oils from a plant matter, per an embodiment of the disclosure herein.

FIG. 1B shows a cross-sectioned side view of the press of an exemplary device for extracting oils from a plant matter, per an embodiment of the disclosure herein.

FIG. 2A shows a cross-sectioned top view of a cooling chamber of an exemplary device for extracting oils from a plant matter, per an embodiment of the disclosure herein.

FIG. 2B shows a cross-sectioned top view of a cooling chamber of an exemplary for extracting oils from a plant matter, per another embodiment of the disclosure herein.

FIG. 3 shows a flow chart of an exemplary method for extracting an organic oil or organic oils from a plant matter, per an embodiment of the disclosure herein.

### DETAILED DESCRIPTION OF THE DISCLOSURE

Provided herein are methods, systems, and devices for extracting oils from organic matter.

The present disclosure may be applied as a standalone method, device or system, or as part of an integrated manufacturing or materials (e.g., chemicals) processing system. It shall be understood that different aspects of the disclosure can be appreciated individually, collectively, or in combination with each other.

Reference will now be made to the figures. It will be appreciated that the figures and features therein are not necessarily drawn to scale. The schematic illustrations, images, formulas, charts, and graphs referred to herein represent fabricated exemplary devices that serve as a representation of the appearance, characteristics, and functionality of the devices produced by the exemplary methods described herein.

The methods, systems, and devices for extracting oils from organic matter, described herein, may play an important role in one or more applications or areas, such as, for example for culinary purposes, for medicinal purposes, for chemical extraction purposes, or for industrial purposes.

Devices for Extracting Oils from a Plant Matter

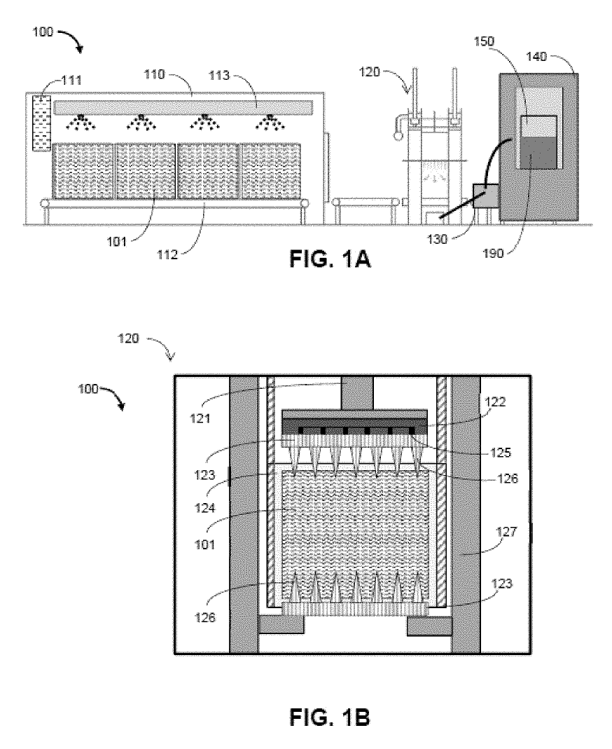

One aspect provided herein is a device for extracting oils from organic matter. In some embodiments, per FIG. 1A, a device 100 for extracting oils 190 from an organic matter 101 comprises, a press 120, a filter 130, a distiller 140, and an oil container 150.

In some embodiments, the device 100 further comprises a cooling chamber 110, wherein the cooling chamber 110 comprises a refrigerator member 111 and a conveyor member 112. In some embodiments, the conveyor member 112 comprises a conveyor member, a slide, a roller, a crane, a forklift, a ramp, a vehicle, a lift, or any combination thereof. In some embodiments, the cooling chamber 110 further comprises a first solvent dispensing member 113. In some embodiments, the first solvent dispersing member 113 comprises a nozzle, a dripper, a hose, a sprinkler, a faucet, a vent, or any combination thereof. In some embodiments, the first solvent dispersing member 113 disperses a combination of a first solvent and air. In some embodiments, the cooling chamber 110 is insulated. In some embodiments, the cooling chamber 110 further comprises at least one of a first solvent dispersing member, a sealing door, a vacuum, and a regulated air intake

In some embodiments, per FIG. 1B the press 120 comprises a compaction member 121, a compaction plate 122, a sieve 123, a second solvent dispersing member 125, an encapsulating member 124, and a frame 127. In some embodiments, the compaction plate 122 comprises a protrusion 126. In some embodiments, the second solvent dispersing member 125 comprises a nozzle, a dripper, a hose, a sprinkler, a faucet, a vent, or any combination thereof. In some embodiments, the sieve 123 comprises a protrusion 126. In some embodiments, the second solvent dispersing member 125 disperses a combination of the first solvent and air. In some embodiments, the press 120 comprises one sieve 123 attached to the compaction plate 122, and one sieve 123 rigidly attached to the frame 127. In some embodiments either the sieve 123 attached to the compaction plate 122 or the sieve 123 attached to the frame 127, comprises or lacks a protrusion 126. In some embodiments, the encapsulating member 124 is configured to temporarily surround the plant matter 101. In some embodiments, the encapsulating member 124 is configured to fully encapsulate the shape of the plant matter 101. In some embodiments, the shape of the encapsulating member 124 comprises a cylinder, a cube, any other polygonal prism, or any combination thereof. In some embodiments, the encapsulating member 124 is configured to encapsulate the plant matter 101 from above, below, a side, or any combination thereof. In some embodiments, the compaction member 121 comprises an electrical compaction member, a pneumatic compaction member, a hydraulic compaction member, or any combination thereof. In some embodiments, the compaction plate 122 and the second solvent dispersing member 125 comprise a single component. In some embodiments, the compaction plate 122 and the compaction member 121 comprise a single component

In some embodiments, the filter 130 has a pore size of about 0.1 μm to about 10 μm. In some embodiments, the distiller 140 comprises a vacuum distiller, a molecular distiller, a steam distiller, an evaporation distiller, or any combination thereof. In some embodiments the oil container 150 retains the organic oil 190. In some embodiments the oil container 150 comprises a liquid conveyor member, wherein the liquid conveyor member transfers the organic oil 190.

In some embodiments, the device 100 further comprises a grinder, wherein the grinder is configured to reduce a size of the plant matter 101. In some embodiments, the grinder comprises a blade, a grinding wheel, a saw, a disk, a wire cutter, or any combination thereof. In some embodiments, the device 100 further comprises a vaporizing member, wherein the vaporizing member is configured to vaporize the organic oil 190. In some embodiments, the device 100 is configured to be mobile, wherein the device can be operated on the field where the plant matter 101 is produced.

In one exemplary method for employing the device 100 herein, the plant matter 101 is loaded onto the conveyor member 112 within the cooling chamber 110 whereafter the first solvent dispensing member 113 dispenses a first solvent onto the plant matter 101. Once the plant matter 101 reaches a certain temperature, it is transferred into the press 120, wherein the encapsulating member 124 moves downwards, upwards, inwards, or any combination thereof, to surround the plant matter 101, the compaction member 121 presses the compaction plate 122, the sieve 123, and the protrusions 126 into and compresses the plant matter 100, while the second solvent dispersing member 125 disperses the second solvent through the sieve 123 and throughout the plant matter. In some embodiments, the processes of compressing and dispersing occur simultaneously or sequentially in any order. Thereafter, the organic oil 190 extracted from the plant matter 100 is transferred through the filter 130 and the distiller 140 to form the organic oil 190, which can be stored in the container 150.

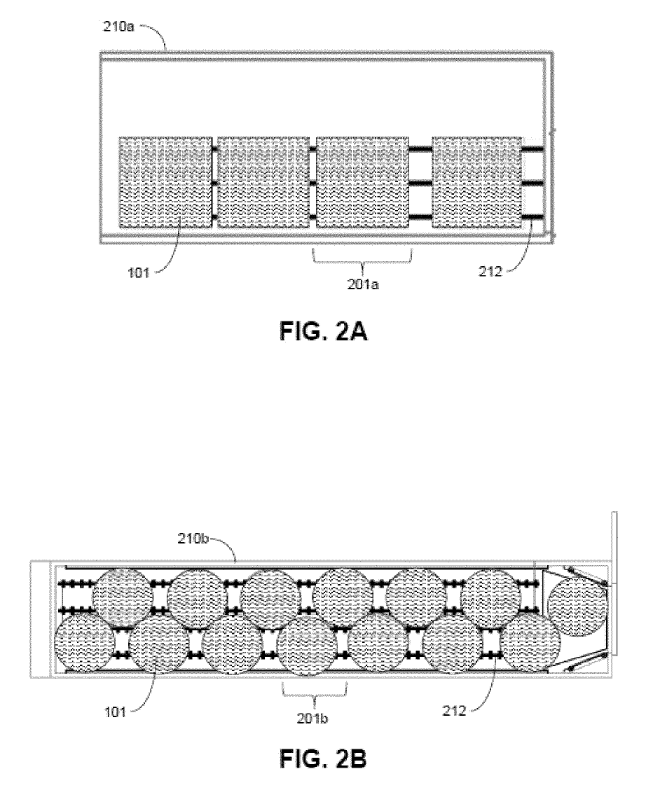

In some embodiments, per FIGS. 2A and 2B, the form of the plant matter comprises a bale 201a 201b or a bundle. In some embodiments the bail comprises plant matter that is packed closely or loosely together. In some embodiments, the shape of each bale 201a 201b comprises a cube or a cylinder, respectively. In a first embodiment of the cooling member 210a, per FIG. 2A, the cooling member 210a is configured to contain and cool two or more bales 201a of plant matter 101, wherein the conveyor member 212 is configured to convey the two or more bales 201a of the plant matter 101, in a single row. In a first embodiment of the cooling member 210b, per FIG. 2B, the cooling member 210b is configured to contain and cool two or more bales 201b of the plant matter 101, wherein the conveyor member 212 is configured to convey the two or more bales 201b of plant matter 101 in two rows. In some embodiments, each bale has a weight of about 100 kg to about 400 kg. In some embodiments, each bail has a diameter of about 0.5 meters to about 4 meters. In some embodiments, each bail has a height of about 0.5 meters to about 4 meters.

Methods and Systems for Extracting Oils from a Plant Matter

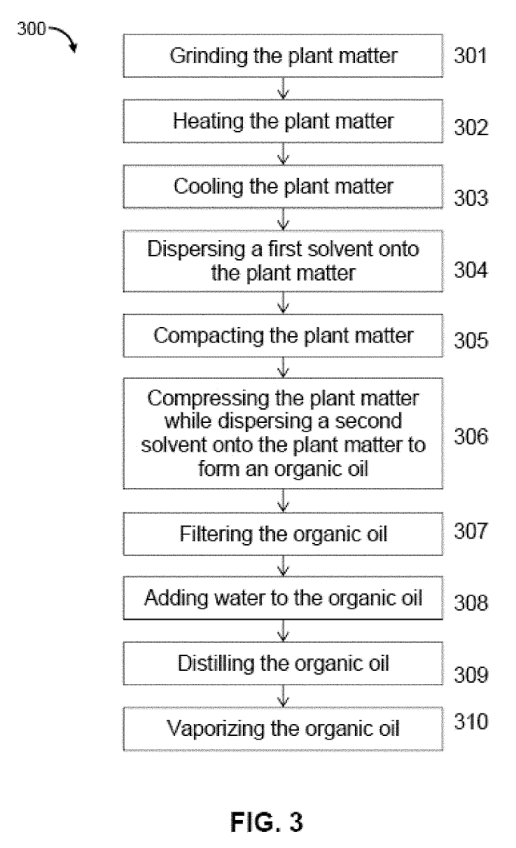

Aspects provided herein are methods and systems for extracting oils from organic matter. In some embodiments, an exemplary method for extracting an organic oil or a combination of organic oils from a plant matter 300, per FIG. 3, comprises cooling the plant matter 303, and compressing the plant matter while dispersing a second solvent onto the plant matter 306. In some embodiments, the method 300 further comprises one or more processes comprising grinding the plant matter 301, heating the plant matter 302, compacting the plant matter 305, filtering the organic oil 307, adding water to the organic oil 308, distilling the organic oil 309, and vaporizing the organic oil 310. In some embodiments, one or more of the processes described herein are repeated or omitted. In some embodiments the order of the processes described herein is altered. In some embodiments, the method further comprises dispersing a first solvent onto the plant matter 304.

In some embodiments, grinding the plant matter 301 reduces the size of the plant matter. In some embodiments, grinding the plant matter 301 is performed by a grinder, wherein the grinder comprises a blade, a grinding wheel, a saw, a disk, a wire cutter, or any combination thereof.

In some embodiments, heating the plant matter 302 comprises heating the plant matter to a temperature of about 50° C. to about 200° C. In some embodiments, heating the plant matter 302 comprises heating the plant matter for a period of time of about 1 hour to about 24 hours.

In some embodiments, cooling the plant matter 303 comprises cooling the plant matter to a temperature of about −125° C. to about 0° C. In some embodiments, cooling the plant matter 303 comprises cooling the plant matter to a temperature of most about 0° C.

In some embodiments, dispersing a first solvent onto the plant matter 304 comprises dispersing a first solvent comprising ethanol, an alcohol, water, formic acid, butanol, pentane, isopropanol, propanol, propylene, methanol, acetic acid, hexanole, or any combination thereof. In some embodiments, the mass of the plant matter is greater than the mass of the first solvent by a factor of about 1.01 to about 100. In some embodiments, dispersing a first solvent onto the plant matter 304 comprises dispersing the first solvent onto the plant matter at a pressure of about 1 bar to about 200 bar. In some embodiments, dispersing a first solvent onto the plant matter 304 comprises dispersing the first solvent onto the plant matter at a pressure of at least about 1 bar. In some embodiments, dispersing a first solvent onto the plant matter 304 comprises dispersing the first solvent onto the plant matter over a period of time of at least about 2 seconds. In some embodiments, dispersing a first solvent onto the plant matter 304 comprises dispersing the first solvent onto the plant matter over a period of time of about 2 seconds to about 24 hours. In some embodiments, compressing the plant matter 304 comprises compressing the plant matter over a period of time of about 2 seconds to about 60 minutes.

In some embodiments, compressing the plant matter while dispersing a second solvent onto the plant matter 306 reduces the volume of the plant matter by about 1% to about 95%. In some embodiments, compressing the plant matter while dispersing a second solvent onto the plant matter 306 comprises dispersing a second solvent comprising ethanol, an alcohol, water, formic acid, butanol, pentane, isopropanol, propanol, propylene, methanol, acetic acid, hexanole, or any combination thereof. In some embodiments, the mass of the plant matter is greater than the mass of the second solvent by a factor of about 1.01 to about 100. In some embodiments, dispersing a second solvent onto the plant matter 306 comprises dispersing the second solvent onto the plant matter at a pressure of about 1 bar to about 200 bar. In some embodiments, dispersing a second solvent onto the plant matter 306 comprises dispersing the second solvent onto the plant matter at a pressure of at least about 1 bar. In some embodiments, dispersing a second solvent onto the plant matter 306 comprises dispersing the second solvent onto the plant matter over a period of time of at least about 2 seconds. In some embodiments, dispersing a second solvent onto the plant matter 306 comprises dispersing the second solvent onto the plant matter over a period of time of about 2 seconds to about 24 hours. In some embodiments, a portion of the first solvent or the second solvent is recycled and reused to extract oil from subsequent batches of plant matter. In some embodiments, compacting the plant matter reduces the volume of the plant matter.

In some embodiments, filtering the organic oil 307 comprises filtering the organic oil through a filter with a pore size of about 0.1 μm to about 20 μm. In some embodiments, the volume of the organic oil is greater than the volume of the water by a factor of about 1.1 to about 100.

In some embodiments, distilling the organic oil 309 comprises vacuum distillation, molecular distillation, steam distillation, evaporation, or any combination thereof. In some embodiments, distilling the organic oil 309 comprises evaporation, wherein the evaporation comprises heating the organic oil at a temperature of about 50° C. to about 100° C., for a period of time of about 0.1 hours to about 160 hours. In some embodiments, distilling the oil removes a portion of water in the oil, a portion of the first solvent in the oil, a portion of the second solvent in the oil, or any combination thereof.

In some embodiments, vaporizing the organic oil 311 comprises ice drying, air pressure drying, convection drying, conduction drying, or any combination thereof. In some embodiments, the method herein further comprises drying the plant matter before grinding the plant matter 301 or heating the plant matter 302.

In some embodiments, the plant matter comprises a coconut, corn, cottonseed, olive, palm, peanut, rapeseed, safflower, sesame, soybean, cannabis, sunflower, nut, seed, fruit, vegetable, citrus, gourd, melon, pumpkin, squash, algae, hemp, any other, or any combination thereof.

In some embodiments the organic oil or the combination of organic oils comprises coconut oil, corn oil, cottonseed oil, olive oil, palm oil, peanut oil, rapeseed oil, safflower oil, sesame oil, soybean organic oil, sunflower oil, nut oil, seed oil, fruit oil, vegetable oil, citrus oil, gourd oil, melon oil, pumpkin oil, squash oil, algal oil, hemp oil, cannabis oil, any other oil, or any combination thereof.

In some embodiments, the method herein further comprises separating the organic oil or the combination of organic oils into two or more organic oils, using any currently available separation means.

### Advantages of the Devices, Methods, and Systems Herein

In some embodiments, an advantage of the devices, methods, and systems disclosed herein is that the devices, methods, and systems herein are more economical to operate, because CO₂ may not be required to extract the organic oil, because less solvent is wasted, because the raw materials may not have to be dried before the organic oil is extracted, because devices, methods, and systems are configured for continuous production, because the cooling process may only comprise a single cooling process, because grinding the plant matter may not be required, because the devices, methods, and systems are configured to be mobile to eliminate unnecessary transportation of the plant matter, because the cooling process may remove sufficient wax from the plant matter to eliminate the need for additional de-waxing processes, and because eliminating or reducing storage times between harvesting and processing reduces production losses due to mold, yeast or pests. In some embodiments, grinding the plant matter is not required because the protrusions on the sieve allow the solvent to access a large surface area.

In some embodiments, an additional advantage of the method disclosed herein is that the devices, methods, and systems are more environmentally friendly, because CO₂ may not be required to extract the organic oil, because the first solvent and the second solvent may be non-toxic, and because the first solvent and the second solvent may be recycled from batch to batch.

In some embodiments, an additional advantage of the devices, methods, and systems disclosed herein is that the devices, methods, and systems herein produce a higher quality oil, because the cooling process is configured to remove more wax, because the devices, methods, and systems are configured to be mobile, to allow for production of oil from field-fresh plant matter, and because eliminating or reducing storage times between harvesting and processing reduces mold, yeast or pest damage. In some embodiments, the devices, methods, and systems disclosed herein are further configured to produce a high quality organic oil, or a combination of high quality organic oils, because desired chemical components such as terpenes or/and acid are not removed during vaporization.

### EXAMPLES

In one exemplary embodiment, per Table 1, five samples of oil were produced from a cannabis plant matter, wherein method parameters comprising the mass and volume of the plant matter, the mass and volume of the solvent, the method temperatures, the time periods, and the use of the water separation process, were altered and tuned to produce an array of cannabis oils.

TABLE 1

| | | TEST NUMBER | | | | |
|---|---|---|---|---|---|---|
| Variables | Unit | 1 | 2 | 3 | 4 | 5 |
| Mass of Plant Matter | g | 70 | 100 | 100 | 100 | 135 |
| Volume of Plant Matter | ml | 120 | 130 | 130 | 130 | 160 |
| Volume of Solvent | ml | 1100 | 500 | 500 | 400 | 500 |
| Solvent Concentration | % | 96 | 96 | 96 | 96 | 96 |
| Initial Plant Matter Temp | ° C. | −28 | −28 | −26 | −25 | −28 |
| Process Plant Matter Temp | ° C. | −17.5 | −15 | −15 | 25 | −15 |
| Dispersing Pressure | bar | 0 | 6 | 6 | 0 | 8 |
| Dispersing Time | min | 0.5 | 20 | 20 | 0 | 20 |
| Mass of Extracted Liquid | g | 700 | 195 | 167 | 60 | 240 |
| Volume of Extracted Liquid | ml | 1000 | 250 | 210 | 100 | 300 |
| Evaporation Temp | ° C. | 74-80 | 73-80 | 73-80 | 73-80 | 73-80 |
| Evaporation Time | hours | 3 | 2 | 0.75 | 0 | 2 |
| Mass of Evapored Liquid | g | 100 | 50 | 20 | 0 | 40 |
| Volume of Evapored Liquid | ml | 115 | 60 | 23 | 0 | 56 |
| Water Separation Temp | ° C. | 3 | 3 | 3 | 2 | 3 |
| Water Separation Time | hours | 72 | 72 | 72 | 72 | 72 |
| Mass of Oil | g | 7 | 6 | <1 | <1 | <1 |
| Volume of Oil | ml | <1 | <1 | <1 | <1 | <1 |

Another example of the method provided herein comprises a method for forming a hemp-based oil comprising freezing the plant matter and coarsely or finely grinding the plant matter to break the unity of the raw material, or compressing the plant matter to minimize stickiness and the energy consumption required to create a smooth coarse homogenous hemp nib pool. In some embodiments, the plant matter is cut at the same temperature at which it was frozen. In some embodiments the plant matter is then fed into a channel, hose, or chamber under pressure or ambient conditions.

In some embodiments, the chamber or channel comprises a mechanism to spray or pump ethanol, or a similar non-toxic or toxic solvent such as butanol, propylene, or propylene hash oil (PHO), through the plant matter at pressure of about 1 bar to about 100 bar. In another embodiment, the plant material is pumped through a container of ethanol. In some embodiments the ethanol is maintained at a temperature of about −15 to about −70 Celsius and is applied over a duration of several hours or a short period of time depending on the pressure the desired solution viscosity and concentration. The distilled ethanol may be reused with the system to be used as a solvent for the next batch of the raw material

### Terms and Definitions

Unless otherwise defined, all technical terms used herein have the same meaning as commonly understood by one of ordinary skill in the art to which this disclosure belongs. As used in this specification and the appended claims, the singular forms “a,” “an,” and “the” include plural references unless the context clearly dictates otherwise. Any reference to “or” herein is intended to encompass “and/or” unless otherwise stated.

As used herein, and unless otherwise specified, the term plant matter refers to any organic material that is not derived from an animal.

As used herein, and unless otherwise specified, the organic oil refers to oil produced from an organic material that is not derived from an animal.

As used herein, and unless otherwise specified, the term distiller or distillation refers an apparatus for, or a process of, separating component or substances within a liquid mixture by selective evaporation and condensation.

As used herein, and unless otherwise specified, the term sieve refers to any organic material that is not derived from an animal.

As used herein, and unless otherwise specified, the term sieve refers to an instrument with a meshed or perforated bottom, used for separating coarse from fine parts of loose matter.

While preferable embodiments have been shown and described herein, it will be obvious to those skilled in the art that such embodiments are provided by way of example only. Numerous variations, changes, and substitutions will now occur to those skilled in the art. It should be understood that various alternatives to the embodiments described herein may be employed.

As used herein, and unless otherwise specified, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about” or “approximately” means within 30 minutes, 25 minutes, 20 minutes, 15 minutes, 10 minutes, 9 minutes, 8 minutes, 7 minutes, 6 minutes, 5 minutes, 4 minutes, 3 minutes, 2 minutes, 1 minutes, 0.5 minutes, 0.1 minutes, or 0.05 minutes of a given value or range. In certain embodiments, the term “about” or “approximately” means within 30 hours, 25 hours, 20 hours, 15 hours, 10 hours, 9 hours, 8 hours, 7 hours, 6 hours, 5 hours, 4 hours, 3 hours, 2 hours, 1 hours, 0.5 hours, 0.1 hours, or 0.05 hours of a given value or range. In certain embodiments, the term “about” or “approximately” means within 30 degrees, 25 degrees, 20 degrees, 15 degrees, 10 degrees, 9 degrees, 8 degrees, 7 degrees, 6 degrees, 5 degrees, 4 degrees, 3 degrees, 2 degrees, 1 degrees, 0.5 degrees, 0.1 degrees, or 0.05 degrees of a given value or range. In certain embodiments, the term “about” or “approximately” means within 30 bar, 25 bar, 20 bar, 15 bar, 10 bar, 9 bar, 8 bar, 7 bar, 6 bar, 5 bar, 4 bar, 3 bar, 2 bar, 1 bar, 0.5 bar, 0.1 bar, or 0.05 bar of a given value or range.

What is claimed is:

1. A device for producing an organic oil from a plant matter comprising:
a) a press which extracts the organic oil from the plant matter, the press comprising:
a compaction member;
a compaction plate;
a second solvent dispersing member;
a sieve; and
an encapsulating member;
b) a filter, wherein the filter is configured to filter:
a first solvent;
a second solvent; or
the organic oil;
c) a distiller; and
d) at least one of:
an oil container, wherein the oil container retains a volume of the organic oil; or
an oil conveyor, wherein the oil conveyor transfers a volume of the organic oil.

2. The device of claim 1, further comprising a cooling chamber which reduces a temperature of the plant matter, the cooling chamber comprising:
a) a refrigerator member; and
b) a conveyor member.

3. The device of claim 2, wherein the refrigerator member is configured to maintain an air temperature inside the cooling chamber of about −125° C. to about 0° C.

4. The device of claim 2, wherein the cooling chamber further comprises at least one of a first solvent dispersing member, a sealing door, a vacuum, and a regulated air intake

5. The device of claim 4, wherein at least one of the first solvent dispersing member and the second solvent dispersing member comprises a nozzle, a dripper, a hose, a sprinkler, a faucet, a vent, or any combination thereof.

6. The device of claim 1, wherein the sieve comprises a plurality of protrusions.

7. The device of claim 1, wherein the encapsulating member is configured to temporarily surround the plant matter.

8. The device of claim 1, wherein the filter has a pore size of about 0.1 μm to about 20 μm.

9. The device of claim 1, further comprising at least one of:
a) a grinder, wherein the grinder is configured to reduce a size of the plant matter;
b) a vaporizing member, wherein the vaporizing member is configured to vaporize at least a portion of the organic oil; and
c) a heating member.

10. The device of claim 1, configured to be mobile.

11. A method for extracting an organic oil from a plant matter comprising:
a) cooling the plant matter; and
b) compressing the plant matter while dispersing a second solvent onto the plant matter.

12. The method of claim 11, wherein the plant matter is cooled to a temperature of about −125° C. to about 0° C.

13. The method of claim 11, wherein the plant matter is cooled to a temperature of at most about 0° C.

14. The method of claim 11, further comprising dispersing a first solvent onto the plant matter before or during the process of cooling the plant matter.

15. The method of claim 14, wherein the mass of the plant matter is greater than the mass of the first solvent or the second solvent by a factor of about 1.01 to about 100.

16. The method of claim 14, wherein at least one of the first solvent and the second solvent comprises ethanol, an alcohol, water, formic acid, butanol, pentane, isopropanol, propanol, propylene, methanol, acetic acid, hexanole, or any combination thereof.

17. The method of claim 14, wherein at least one of the dispersing of the first solvent onto the plant matter, and the dispersing of the second solvent onto the plant matter occurs at a pressure of about 1 bar to about 200 bar.

18. The method of claim 14, wherein at least one of the dispersing of the first solvent onto the plant matter, and the dispersing of the second solvent onto the plant matter occurs over a period of time of at least about 2 seconds.

19. The method of claim 10, wherein compressing the plant matter occurs over a period of time of at least about 2 seconds.

20. The method of claim 10, further comprising heating the plant matter before the cooling of the plant matter.
